(19) United States
(12) Patent Application Publication
(10) Pub. No.: US 2005/0175588 A1
(43) Pub. Date: Aug. 11, 2005

(54) Production of a protein delivery system for in vivo therapeutic treatment

(76) Inventor: Joseph D Mosca, Ellicott City, MD (US)

Correspondence Address: Joseph D Mosia, 4201 Blue Barrow Road, Ellicott City, MD, 21042, US

(21) Appl. No.: 10/503,755

(22) PCT Filed: Feb. 11, 2003
(86) PCT No.: PCT/US03/04255

Related U.S. Application Data
(60) Provisional application No. 60/356,300, filed on Feb. 11, 2002.

(51) Int. Cl. A61K 48/00; C12N 15/86

(52) U.S. Cl. 424/93.2; 435/456

(57) ABSTRACT

Disclosed is a generalized protein delivery system that (i) contain one or more recombinant molecule(s) that are expressed on a cellular surface and (ii) incorporated and/or associated with a particle that does not require cellular entry for delivery. The recombinant protein is preferably a protein, peptide, and/or antibody. The particle is preferably a virus-like-particle devoid of any intrinsic infectivity. Also disclosed is a method for formation and production of the particles able to carry at least one recombinant molecule. In addition, methods are disclosed for the delivery of the recombinant particles into a mammalian host for the prophylactic therapeutic treatment and/or as a diagnostic reagent for human diseases.

### FIELD OF THE INVENTION

This invention relates to the field of delivery of therapeutic proteins for prophylactic purposes in mammals by the production of a protein delivery system capable of delivery of recombinant proteins, stimulation of biological processes, inactivation of inhibitory chemical and/or biological factors, and scavenging and/or detoxifying any molecule for therapeutic benefit either in vitro or in vivo.

### BACKGROUND OF THE INVENTION

Eukaryotic protein expression systems are frequently employed for the production of recombinant proteins as therapeutics as well as research tools. Most commonly used expression systems are based on stably transfected Chinese hamster ovary (CHO) cells and infection of insect cells by recombinant baculoviruses. Although much success has been associated with protein production in established protein expression systems, numerous obstacles are the source of structural heterogeneity that could lead to functional deficiencies. Comparison of intracellular transport and processing of a recombinant glycoprotein in these systems differ considerably among each other and often different from the native protein. Intracellular proteins in both systems are associated with oligosaccharide protein glycosylation, but are absent or under-represented in sialylated glycoforms, in addition to post-protein glycan processing. Differential post-translational modification of synthesized proteins can profoundly affect three-dimensional structure and biological function.

Sialylation of N-glycans associated with human serum proteins has a central role in determining its circulatory clearance rate. Higher the sialylation, the longer the clearance rate in the mammalian circulation. Clearance times can change from minutes to hours with increased protein sialylation. Although the degree of sialylation may play an important adaptive response during early development, the reduced sialylation during production of recombinant proteins that require sialylation in our present eukaryotic expression systems has made these recombinant products almost useless as therapeutics. Approaches to overcome these innate deficiencies have either exposed recombinant proteins in vitro to exoglycosidases and sialyltransferase or have introduced liver derived beta-galactoside alpha-2,6-sialyltransferase cDNA by gene transfer into cells producing the recombinant protein. The in vitro incorporation of sialic acid into neuraminidase-treated recombinant proteins (developed specifically to allow efficient sialic acid capping of beta-galactose-exposed termini) has been shown to saturate >70% of the theoretical acceptor sites. Similarly, recombinant proteins produced by the gene-modified cells displayed a significantly higher proportion of fully sialylated glycans. Both sialylated recombinant serum proteins made from genetically modified cells and those subjected to extensive sialylation in vitro exhibited increased circulatory retention times approaching the degree of sialylation and retention times found on proteins purified from native tissue sources. Although these approaches accomplish the desired result, they are cumbersome and time consuming. They are costly and yields are low. Liver cells are the in vivo source of many of these highly sialylated glycoproteins containing sialyltransferase that are involved in the sialylation of O-glycosidically linked carbohydrate chains on serum glycoproteins. This observation suggests that hepatic cells rather than Chinese hamster ovary cells or baculovirus would be the cell line of choice when expressing these serum proteins. Thus, it can be concluded that expression of these recombinant proteins from the same cells that produce these proteins in vivo may improve their pharmacokinetic behavior and their prophylactic therapeutic usefulness either in vitro or in vivo.

In addition to sialylation and similar post-translational protein modifications, another innate deficiency in our present eukaryotic protein expression systems is the ability to multimerize monomeric forms of expressed recombinant proteins. In most if not all cases, monomers of expressed proteins require higher forms of structure in order to carrier out their intended function. Multimerization of protein monomers into dimeric, trimeric, or tetrameric structures rely on protein-protein interactions and in some cases disulfide-linkage. In some cases these interactions are intrinsic to the molecule; in others, cellular encoded proteins facilitate these oligomeric super-structures. There is an increasing awareness from a spectrum of genetic deficiencies that mutations in a “linking or anchoring” protein and not in the specific gene itself is responsible for various congenital syndromes. These deficiencies are caused by an uncoordinated expression of protein subunits and linking/anchoring proteins that normally determines the pattern of molecular forms, which in turn determines the localization and functionality of the resulting protein.

Thus, our present state of the art for eukaryotic protein expression systems fail in at least two post-translational modifications that are required for the high level of expression of recombinant glycoproteins whose sialic acid content is important for their function and pharmacokinetic behavior—the level and nature of N-glycan capping and subunit assembly.

As the state of the art in molecular recognition of biowarfare agents and other pathogens improves, there is an increasing need to develop quick acting and efficient therapeutic recombinant bioscavengers for defense against these chemical and biological agents. The biocatalytic destruction of organophosphates has become an important focus area and efficient technologies are being sought for counteracting chemical weapons to afford protection against nerve agents and pesticide poisoning. Novel methods have been advanced using enzymes hydrolyzing organophosphates as potential catalytic scavengers against organophosphate poisoning such as organophosphorous hydrolase from Pseudomonas diminuta, carboxylesterase, and the role of phosphotriesterases in the detoxification of organophosphorus compounds. With the recognition that broad-spectrum organophosphorus insecticide were designed to produce acute cholinergic effects by inhibition of acetylcholinesterase and the knowledge that organophosphorus insecticide exposure exhibits only moderate acute toxicity in mammalian species due to rapid detoxification of the active metabolite by acetylcholinesterase. Research has focused on the use of native cholinesterases as a mode of treatment to prevent organophosphate toxicity. Research in this area demonstrated the ability of native cholinesterases as an effective mode of pretreatment to prevent organophosphate toxicity in mice and rhesus monkeys. Tissue-derived cholinesterases were compared to recombinant cholinesterases after intravenous injection into mice and rhesus monkeys, illustrating the reduced circulatory half-life of the recombinant protein. The summary of a large body of research points to the innate deficiencies in sialylation and monomers assembly in tetrameric active protein subunits in our present eukaryotic expression systems as the underlining reasons for the observed instability in vivo. Until these obstacles are overcome, recombinant approaches to therapeutic detoxification of chemical and biological agents will not be a realistic prevention strategy for preventing toxicity. The current, non-recombinant, approach of isolating cholinesterases from natural sources of human plasma and other blood by-products is impractical due to the large quantities required and the potential biohazard associated with the presence of known (HIV-1, hepatitis, prions, etc.) and yet to be discovered potential pathogen present in pooled human-derived tissue products. Recombinant molecular technology is the only practical way to accomplish the goal to attain large quantities of human cholinesterases. We need chemical/biological defense agents displaying a high stability upon long-term storage in order to define its therapeutic capacity in vivo, its pharmaceutical properties for clinical trials, and if the results are successful, for general use in detoxification of organic compounds. Thus, there is a requirement for a generic technology that has versatility and economy-of-scale that would allow production and purification of kilogram quantities of any number of specific bioscavengering agents.

In addition to bioscavenging agents, the above discussion relates to any and all in vitro synthesized amino acid containing product. Recognition of proteins, peptides, and antibodies by their biological cellular receptor is dependent on the “decoration” of the primary amino acid sequence with sugars, lipids, and nucleic acids modifications. These modifications that effect biological processes, which are mostly post-translational, are performed within specialized cells through out the mammalian body independent of the molecule being eukaryotic or prokaryotic in origin. The biological processes include cellular and non-cellular, but molecular receptor-mediated binding of an amino acid containing sequence to induce or inhibit differentiation, to stimulate or suppress immune responses, to attract or repel cells and/or biomolecules, to prevent organ and/or host toxicity—basically to influence biological processes in vitro and/or in vivo in a positive way to achieve a favorable therapeutic or preventive outcome in an mammalian host, especially when that mammalian host is human.

Many viruses produce degenerative changes in cells when replicating in susceptible cells in culture in vitro. These characteristic changes are called cytopathic effects and are associated with certain morphologic changes in the host cell. The intracellular sites where the events of viral replication take place vary among the viral families. Enveloped viruses mature by a budding process, although some budding occurs with non-envelope containing viruses. For envelope viruses, viral-specific envelope glycoproteins are inserted into cellular membranes and the viral nucleocapsids then bud through the membrane at these modified sites. In this process, the virus acquires their envelope for infectivity and can also acquire cellular-related molecules. Studies with HIV, Influenza, and Chlamydia have shown viral particles that have incorporated HLA molecules into the mature virus particle. During the infection the cell is destroyed and the virus particles are released into the culture supernatant. The amount of infectious virus present in the cell culture fluid can be titrated and infectivity inactivated by a variety of methods. Although inactivated virus particles have lost the ability to replicate, they maintain their structure and as detailed in this application, they can be used as a scaffold to carry cell surface expressed molecules.

The final step in the lytic cycle of enveloped viruses involves the budding of the newly formed particles from cellular membranes. Studies of viruses that obtain their envelope from the plasma membrane have established the dependence of virus budding on interactions with viral proteins. For the alphavirus—Semliki Forest virus, it has been established that virus budding is strictly dependent on interactions between the transmembrane spike protein and the internal nucleocapsid. In retroviruses, however, interactions between the cytoplasmic tail of external viral protein and the internal viral components are not a prerequisite for virus budding since expression of the gag protein alone is sufficient to drive budding of virus-like-particles. A different mechanism directs the assembly and release of coronavirus particles. The particles assemble at intracellular membranes and expression of viral membrane proteins drives the assembly and budding of virus-like-particles. Overall matrix protein plays a pivotal role in assembly of RNA viruses. The M1 proteins of vesicular stomatitis virus, human parainfluenza virus type 1, and influenza-A has intrinsic budding activity when expressed alone. In fact, similar to retroviruses, interactions between the internal viral component and the cytoplasmic tail of external virus proteins are not an absolute requirement for virus particle formation. Similar to inactivated virus particles, virus-like-particles maintain their structure and as detailed in this application, they can be used as a scaffold to carry cell surface expressed molecules.

### SUMMARY OF THE INVENTION

This invention provides for the formation, production, and in vivo delivery of recombinant molecules for therapeutic and diagnostic purposes. The invention could contain one or more than one molecule or contain native cell surface components from a particular cell type. Molecules preferably include any amino acid moiety-containing molecule, but other molecules captured during the process covered by this invention could be envisioned. Formation of specific molecules could be engineered genetically by molecular biology techniques to be expressed on the surface of cells that alone or together with native molecules on the said cells' surface, forms the essence of the invention. The formation and production of the invention involves the removal of cell surface membrane components as a consequence to the budding of particles from within the cell. The particles could be made of single or multiple components. Components are envisioned to be viral in origin, but could be induced by non-viral methods, or natural to the cell selected host. The invention is preferably for in vivo delivery, but could be used in vitro for induction or maintenance of cellular processes. Processes include, but not limited to, cellular signaling, cellular induction, cellular suppression, cellular attractant, cellular differentiation and/or cellular or molecular scavenging. In vivo delivery could be by intravenous injection, but other routes include but are not limited to oral, suppository, intramuscular, inter-cranial, inter-peritoneal, or directly into mammalian organs, capillaries, ducts, or lymphoid system either alone or associated with biological or non-biological materials or devices. Inter-respiratory devices, cutaneous and topical applications are also envisioned within this invention. In addition the application of the invention as aerosols, creams, puffers, or on surfaces, is included in this invention. Surfaces include, but not limited to, synthetic, non-synthetic, biological, or non-biological matrixes including autologous, allogeneic, and xenogeneic extracellular matrix materials. Therapeutic purposes encompass all procedures and/or processes that result in the improvement or intended improvement of the health and well being of an inflicted human or mammalian host.

In addition to therapeutic protein production, this invention encompasses counter-measures against chemical and biological defense agents using recombinant molecular technology could be envisioned, where the counter-measure is a biological or chemical molecule(s). The counter-measure is preferably a molecule that binds and inactivates a chemical or biological toxin, or inhibits cellular entry of said toxin or biological agent, thereby preventing damage to biological human and mammalian tissues. Although the counter-measure is conceived to be a protein expressed from a eukaryotic cell or protein expression system by a defined nucleic acid sequence(s), it need not be limited to such biological molecules and as a protein, it may require additional post-translational modifications to enable the counter-measure to provide the necessary disabling function(s). The invention is intended for in vivo use in any recipient (human or mammalian) requiring detoxification, although in vitro usages can also be envisioned.

In addition, this invention relates to the prevention of toxicity and/or organ failure do to the accumulation of metabolites and/or deposits due to the nature of the recombinant protein being synthesized from cellular sources foreign to the native molecule and host. In this capacity, the invention is envisioned to produce a biological carrier delivery system where the captured molecule(s) is indistinguishable from the native molecule(s) with the exception of being embedded or attached to a biological carrier (inactivated virus or virus-like-particle). The molecule or molecules would be in the native configuration containing all possibly required modifications that are native to said molecule(s), and as such, contains the innate ability to behave and maintain the same biologically activity as if the molecule(s) were synthesized in vivo. As such the molecule would be native to the host and as such will not cause the accumulation of metabolites that would be toxic to the host, nor result in deposits in organs that could be detrimental to the host. Toxicities and unusual deposits are sometime intrinsic to recombinant material synthesized in either commonly used in vitro systems, especially when these molecules are synthesized in non-mammalian systems like yeast or bacteria.

In one aspect the invention is a bio-scavenger, a receptor or an enzyme. As a bio-scavenger, the invention could bind, chelate, sequestered, and/or inactivate a chemical or biological agent. As a receptor expressed on the surface of a human or mammalian cell that induces particle formation by budding off membrane pieces containing the recombinant receptor, the invention provides a mechanism to bind, chelate, sequester, and potentially clear any biological inhibitor agent(s), molecule(s), or process from a human or mammalian biological body or system. As a receptor, the molecule may need to be modified to make the molecule cell surface expressed-for example addition, removal, and/or replacement of signal peptide, intracellular, transmembrane and/or other molecular domain sequences. As a said receptor, the agent binds, sequesters, and clears the biological inhibitor molecule or toxin as a complex from the body. As an enzyme the agent binds, inactivates by enzymatic cleavage or non-enzymatic hydrolyze to metabolites that are no longer harmful to human or mammalian tissues and/or hasten the removal of the toxin from the host. Inactivation can occur, but not limited to enzymatic cleavage, blocking of reactive moieties, masking of active site(s), sequestering to certain tissues, and/or clearance of the toxin as a bound or unbound complex. In one embodiment of this aspect, the cDNA for a specific biological molecule or molecules is introduced by mechanical, physical, chemical, or viral means into cells capable of high-level cell-surface protein expression. Mechanical, physical, and chemical means include but not limited to electroporation, and/or lipid-mediated, polyethylene glycol, Sendai virus membrane fusion that bypasses the cellular membrane to gain access to the cellular chromatin structure where integration may or may not occur. Viral mediated delivery mechanisms include but not limited to murine leukemia virus (MuLV), adenovirus, adeno-associated virus (AAV), lentivirus, and canarypox vectors. The human or mammalian cells capable of high-level cell-surface protein expression could be any primary, transformed, and/or established cell line of autologous, allogeneic, or xenogeneic nature.

In another aspect the invention provides co-expression of molecules and/or portions of molecules that either facilitates assembly, configuration, conformation, or co-expression of proteins to stimulate one or more than one cellular process at a time. These modifications or co-expressed molecules assist in the final biological activity of the expressed recombinant proteins are covered within the scope of the present invention. Biological activity can be, but not limited to: (i) binding affinity/avidity of inhibitor molecules; (ii) enhancement of enzymatic hydrolyze; (iii) blocking of the inhibitor molecule mode of action; (iv) stabilizing the recombinant protein itself and/or when in a recombinant protein: inhibitor molecular complex; (v) enabling biological activity over extended periods of time in biological fluids; (vi) enhancing clearance and/or removal from the host once the recombinant protein is complex with the inhibitor molecules; (vii) delivery of any molecule(s) for preventative, therapeutic, and/or maintenance purposes; and/or (viii) activation, induction, differentiation, homing and/or attracting cells in vitro and/or in vivo. These biological activity properties could be achieved by, but not limited to: (i) the introduction of a genetic sequences into a cell; (ii) expression of a protein(s) on the cell surface; (iii) expression of molecule(s) that enhance the biological effect; (iv) production of particle release either innate to or induced by the introduction of viral or non-viral components by either mechanical, chemical, and/or viral vector means; (v) harvesting of released particles; (vi) concentration of released particles; (vii) inactivation of release particles; (viii) lyophilization of particles for long-term storage; (ix) exposure of cells, organs, or biological systems in vitro to particle preparation; and/or (x) delivery of particle preparation in vivo by any conceivable route.

In another aspect the invention provides for molecules containing the appropriate post-translational modifications required for human and mammalian proteins found in vivo. This is due to the de novo synthesis of protein molecule(s) within the same cells that the protein is naturally expressed in situ. The invention supplies advantages to the recombinant molecule(s) in that the formation and production of the said molecule is in accordance with, and as close as possible to the naturally expressed molecule. The adherence to the native protein in conformation, configuration, multimerization, and post-translational modifications that include but not limited to glycosylation, polyADPribosylation, and myristylation, improves the functional parameters associated with the said molecule(s). The gene and/or nucleic acid sequence would be either naturally expressed on the surface of the cell or genetically modified by the addition of heterologous sequences to the said sequence in such a way that the said molecule would be cell surface expressed. Cell modification could be by gene transfer. Any number of viral or non-viral vectors or direct delivery methods could introduce any number of genes to express proteins onto the cells' surface. The cell surface expressed protein(s) are captured into particles as the particles are released from the cell. Genes could code for molecules, including but not limited to, enzymes, receptors, receptor ligands, cytokines, growth factors, and antibodies. In addition, the introduced gene(s) could code, but not limited to molecules involved in oocyte and/or cellular differentiation, homeostatic maintenance, and/or initiating intracellular signaling pathways that either enhance or inhibit biological processes.

The present invention describes a protein delivery technology that has demonstrated the ability to incorporate over-expressed proteins into either active or inactive viral particles and/or virus-like-particles, but the invention is not limited to viral induced budding components. The process relies on the biological process of particle release to remove pieces of the cellular membrane while exiting a said host cell. Stable cell lines are loaded with a recombinant protein of interest on the surface of cells by standard molecular biological transfection or transduction techniques. The modified cell may be either (i) infected with a virus that is able to productively induce a lytic or non-lytic infection resulting in virus progeny; (ii) chronically infected and continuously release virus particles; (iii) transfected or transduced with one or more viral component either transiently or permanently expressed that results in particle release; and/or (iv) naturally or artificially induced to release particles of cellular origins capable of capturing molecules expressed at cellular membranes. In all cases, the released particles contain the same over-expressed protein present on the host cells' surface and as such serve as a novel delivery system for recombinant molecules. In this way, single or multiple molecules are expressed with similar native structure to the naturally expressed human or mammalian protein.

The “capture” of a protein on the surface of a particle simplifies the process of synthesizing and purifying recombinant molecules and/or proteins to harvesting virus particles. Thus, in vitro recombinant protein systems can be simplified to purification of viral/non-viral/cellular particles or viral-like-particles using standard generic techniques. At the same time this technology insures proper orientation, conformation, and post-translational modifications of the synthesized protein, since the protein is made de novo. These modifications are not being met for recombinant proteins expressed in standard systems presently in use—like Chinese hamster ovary cells (CHO) and infection of insect cells by recombinant baculoviruses due to intrinsic deficiencies in these systems. To further insure proper post-translational and conformational recombinant protein synthesis, host cells that naturally express the protein could be chosen as the host for in vitro synthesis.

In summary, the present invention describes the utility of a process to deliver and produce recombinant molecules for therapeutic and diagnostic purposes. For therapeutic purposes, the invention is a protein delivery system; for diagnostic purposes, the invention is a method to capture native molecules representing innate structures identical to those found in vivo for measurement of immune responses or as a source of native molecules for purification and diagnostic kit purposes, including but not limited to molecules from hazardous bio-organism. The invention has a wide range of applications, including but not limited to counter-measures against chemical and biological defense agents. The invention provides a method to synthesize, produce, and purify biologically active molecules incorporated in and/or attached to intact particles for therapeutic, diagnostic, reagent and/or protective purposes. The goal of this invention is to bestow biological properties similar to or improved upon that found in natural biological systems. The invention is a biological agent expressed as a particle containing one or more recombinant protein for in vitro and in vivo use to modify, enhance, protect, and/or induce cellular processes.

### BRIEF DESCRIPTION OF THE DRAWINGS

The invention is further described by the accompanying drawings and the description thereof herein, although neither is a limitation of the scope of the invention.

FIG. 1 is a schematic representation of the protein delivery system covered by the present invention. The figure illustrates the introduction of nucleic acid sequences into a specific host cell and the expression of the nucleic acid molecules onto the cell surface of said host cell. In this embodiment, the introduced transcribed and translated nucleic acid sequences expressed on the host cell surface are “captured” upon release of the infectious agent that is used to infect the nucleic acid modified host cell. The figure further illustrates the harvesting, concentration, and inactivation of the infectious agent released particle preparation. In this figure, the protein delivery system is referred to as “Biological Carriers.” The term biological carriers were coined to emphasis both the biological nature of the technology and their ability to carry fully expressed molecules.

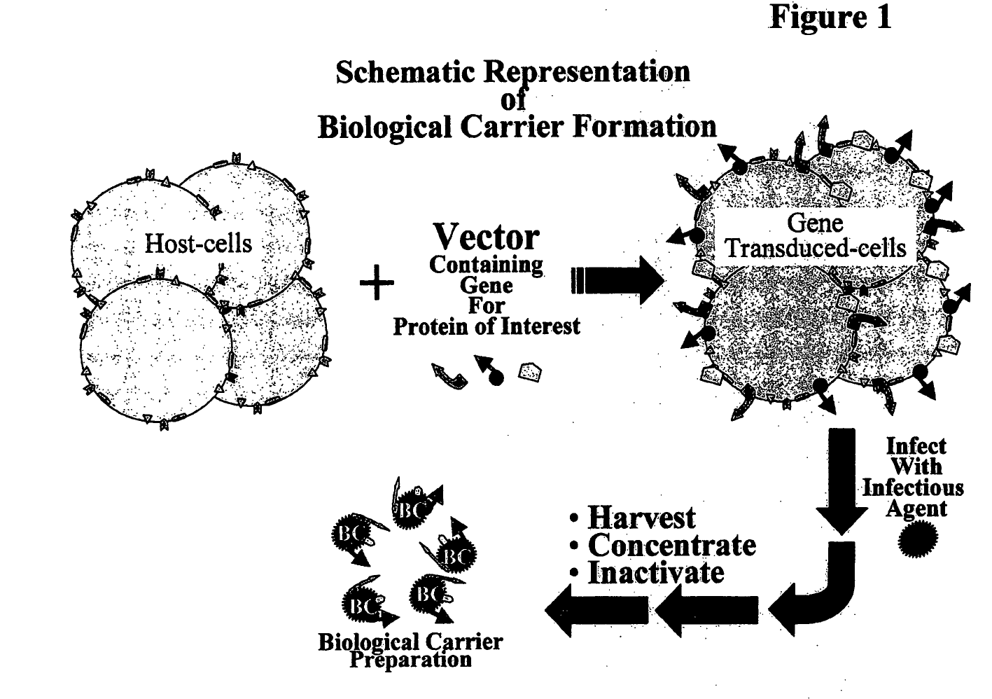

FIG. 2 is a schematic representation of one potential application of the “biological carrier” protein delivery technology—its ability to interact with T-cells to influence the activation and formation of immune cells directed against specific forms of cancer and/or infectious diseases. The diagram shows the particles interacting with T-cells, but these interactions could be with any cell type in vitro or in vivo. The activation is envisioned to be by receptor-mediated signal transduction induced pathways, but mechanisms presently known or unknown may be involved. In this figure, the protein delivery system is referred to as “Biological Carriers.” The term biological carriers were coined to emphasis both the biological nature of the technology and their ability to carry fully expressed molecules.

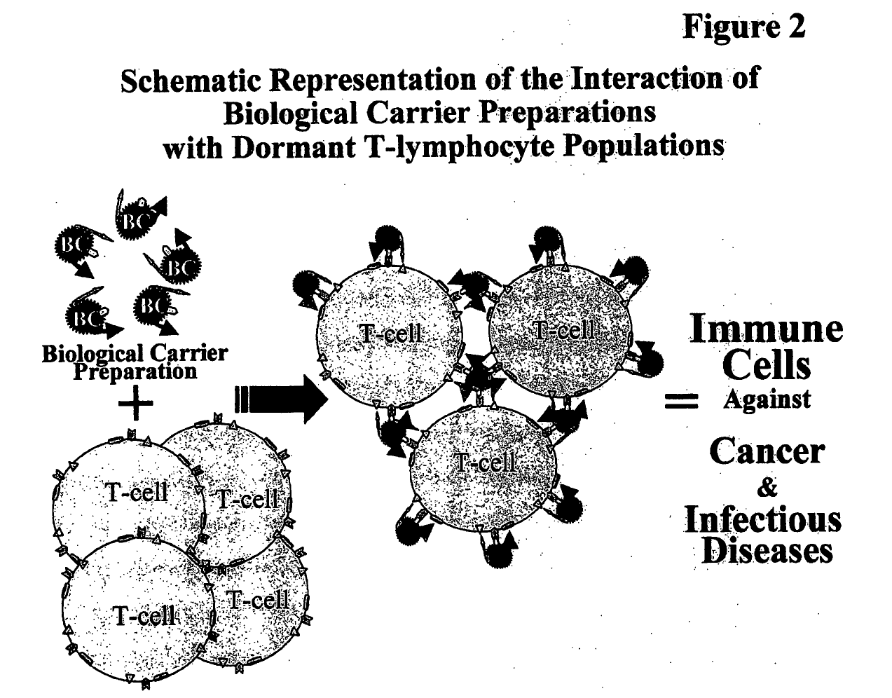

FIG. 3 is a schematic representation of the use of this protein delivery system with respect to both an Acquired Immunodeficiency Syndrome (AIDS) therapeutic and diagnostic reagent. The figure illustrates the formation of particles from a chronically infected cell line where the infectious agent is continuously budding from the cell lines' surface. In the present diagram, the human immunodeficiency virus (HIV), the etiological agent of AIDS is illustrated, but similar cell lines harboring infectious agents or portions of infectious agents could be envisioned. As an AIDS therapeutic, costimulatory molecules are introduced into the chronically-infected cell line, which together with HIV-specific antigens processed as peptides in major histocompatibility (MHC) molecules that are presence on the cell line due to chronic expression of HIV, should be able to stimulate immune responses when contacting the appropriate cells either in vitro or in vivo. As an AIDS diagnostic, the particles could be used as a preparation for the purification of specific viral proteins and/or nucleic acids, in addition to an antigen preparation for detection of immune responses against HIV in humans for analysis of the presence of antibodies against specific viral antigens. Detection could be by ELISA, PCR or Western Blot, but not limited to such detection systems. In addition, the schematic illustrates the “capturing” of cellular surface molecules that are native to said host cell, are also present in the final particle preparation. In this figure, the particles are identified as BC, which refer to Biological Carriers. The term biological carriers were coined to emphasis both the biological nature of the technology and their ability to carry fully expressed molecules.

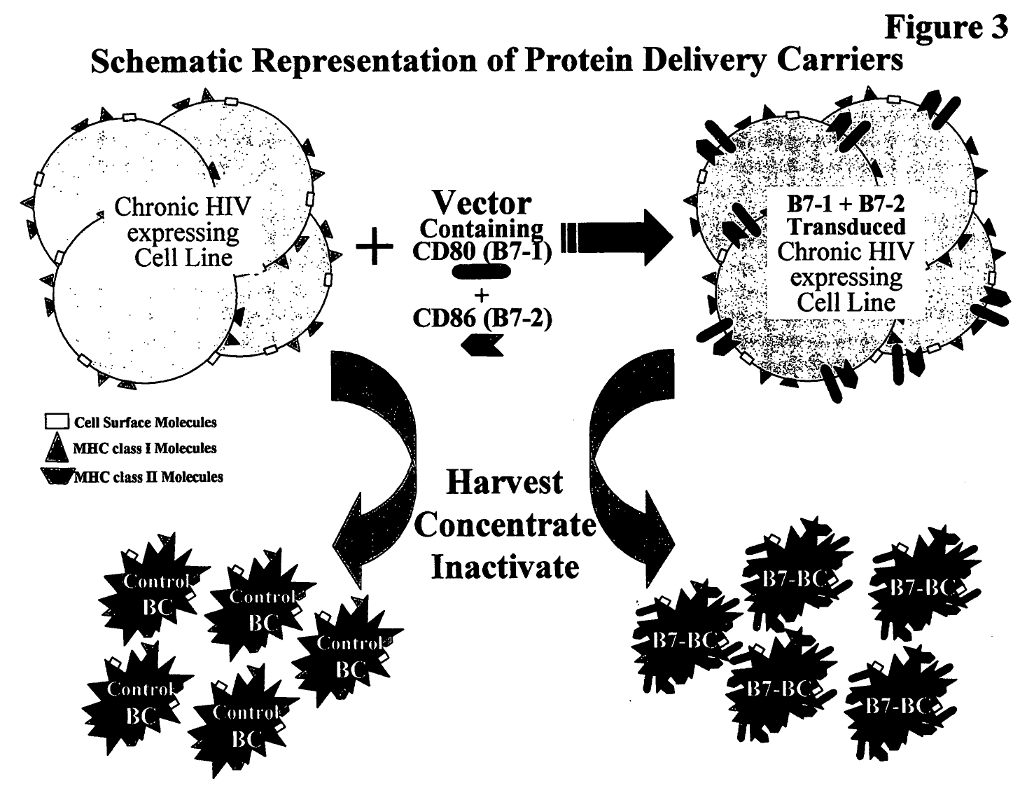

FIG. 4 is a schematic representation for using virus-like-particles (instead of infectious virus particles that are subsequently inactivated) as the vehicle to capture, incorporate and deliver molecules. As an example the M1 Influenza-A matrix protein was used as an illustration, but other viral and/or non-viral components alone or in combination could be envisioned. As a further illustration of the versatility of the invention, HIV or HSV specific antigens are expressed along with costimulatory molecules to induce immune responses. In contrast to FIG. 3, the established chronic cell line constituently expresses particles that are continuously released from the said cell line. As a therapeutic, the specificity of the immune response comes from the specific inclusion and expression of infectious viral antigens—gp160/120 & gag antigens for HIV; gpB2 & gpD for HSV-2. The specific antigens either intact or processed as peptides in major histocompatibility (MHC) molecules together with expressed costimulatory molecules have been shown to elicit corresponding immune responses when binding to the appropriate cells either in vitro or in vivo. As a diagnostic, the particles could be used as a preparation for the purification of specific viral proteins and/or nucleic acids, in addition to an antigen preparation for detection of immune responses against HIV or HSV in humans by the analysis for the presence of antibodies against specific viral antigens. Detection could be by ELISA, Western Blot and/or immune fluorescence, but not limited to such detection systems. Unlike the previous schematics, all the viral antigens and co-stimulatory molecules contain hybrid constructions made between the said molecule and intracellular domain sequences of Influenza-A surface proteins—NA refers to the neuraminidase antigen and HA refers to the hemagglutinin antigen, although the addition of these sequences are not a prerequisite for the inclusion of these said molecules into the released particles. In addition, the schematic illustrates the “capturing” of cellular surface molecules that are native to said host cell, are also present in the final particle preparation. In this figure, the particles are identified as BC, which refer to Biological Carriers. The term biological carriers were coined to emphasis both the biological nature of the technology and their ability to carry fully expressed molecules.

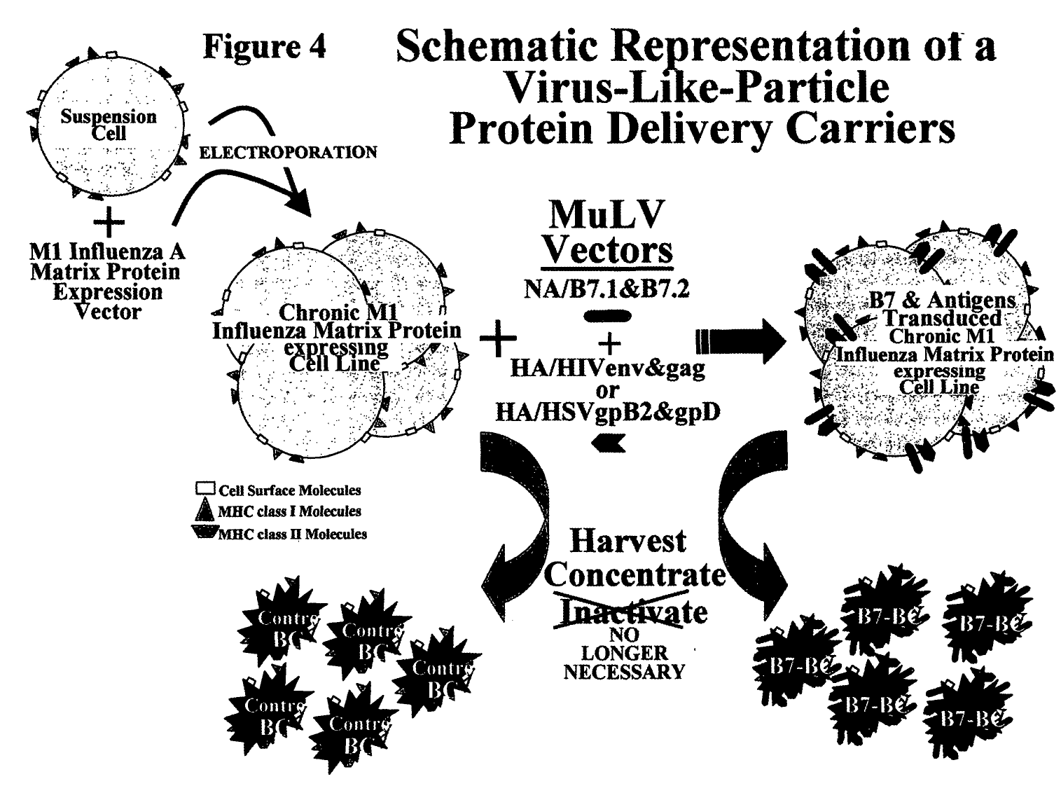

FIG. 5 is a schematic representation for using virus-like-particle release from a continuous particle expressing cell line to incorporate within the released particles the cellular receptor (TEM-8) for the Protective Antigen (PA) protein from the bacterium Bacillus anthracis, the etiologic agent of anthrax. As a therapeutic, the released particles—TEM-8 particles—could be used in vitro or in vivo to bio-scavenge PA protein to prevent cellular and tissue damage within the host either for prophylactic and acute protection. The TEM-8 coding sequence is expressed on the surface of particle budding cells as an in-frame hybrid gene with the Influenza-A hemagglutinin antigen intracellular domain. As a diagnostic, the TEM-8 containing particles could be used, but not limited to, a detection system within an assay to bind anthrax specific antigen.

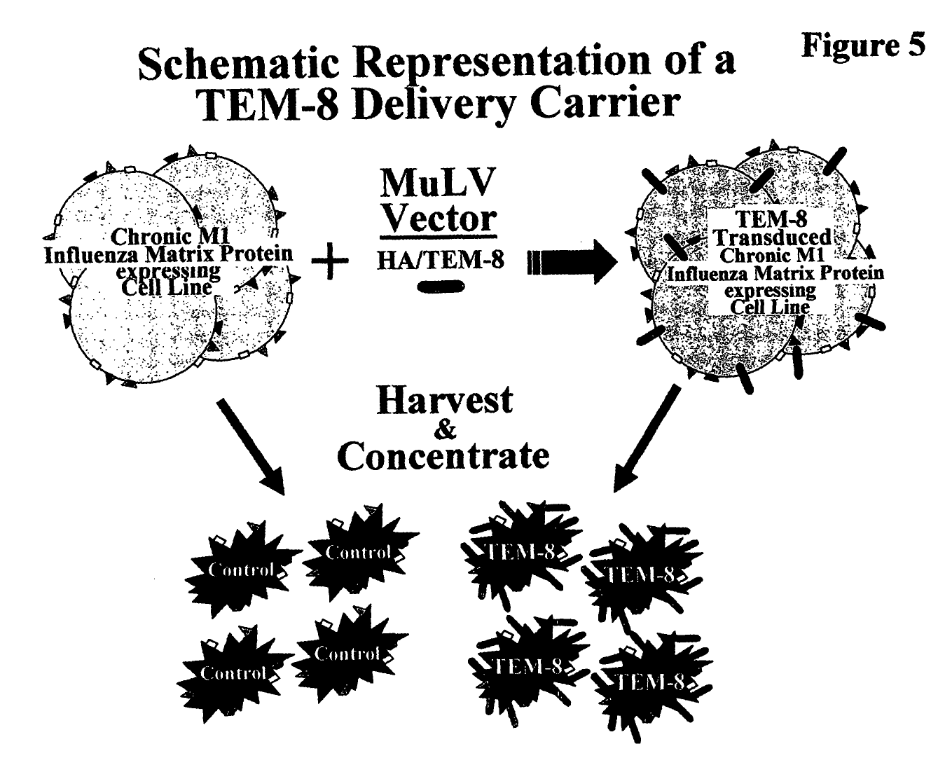

FIG. 6 is a schematic representation for using virus-like-particle release to incorporate either costimulatory molecules alone or costimulatory molecules together with a hybrid genetic construction containing the ecto- or extra-cellular domain sequence of the Protective Antigen (PA) protein from the bacterium Bacillus anthracis, the etiologic agent of anthrax. In this figure the particles are made from a single viral component, the M1 Influenza-A matrix protein, but could be made from multiple components that are viral, non-viral, or innate to its source cell line. As a therapeutic, the PA antigen either intact or processed as peptides in major histocompatibility (MHC) molecules together with expressed costimulatory molecules will elicit corresponding immune responses involving both the humoral and cell-mediated arms of the mammalian immune system, including mechanisms presently known or unknown to eliminate and/or protect by either in vitro or in vivo mechanisms the host.

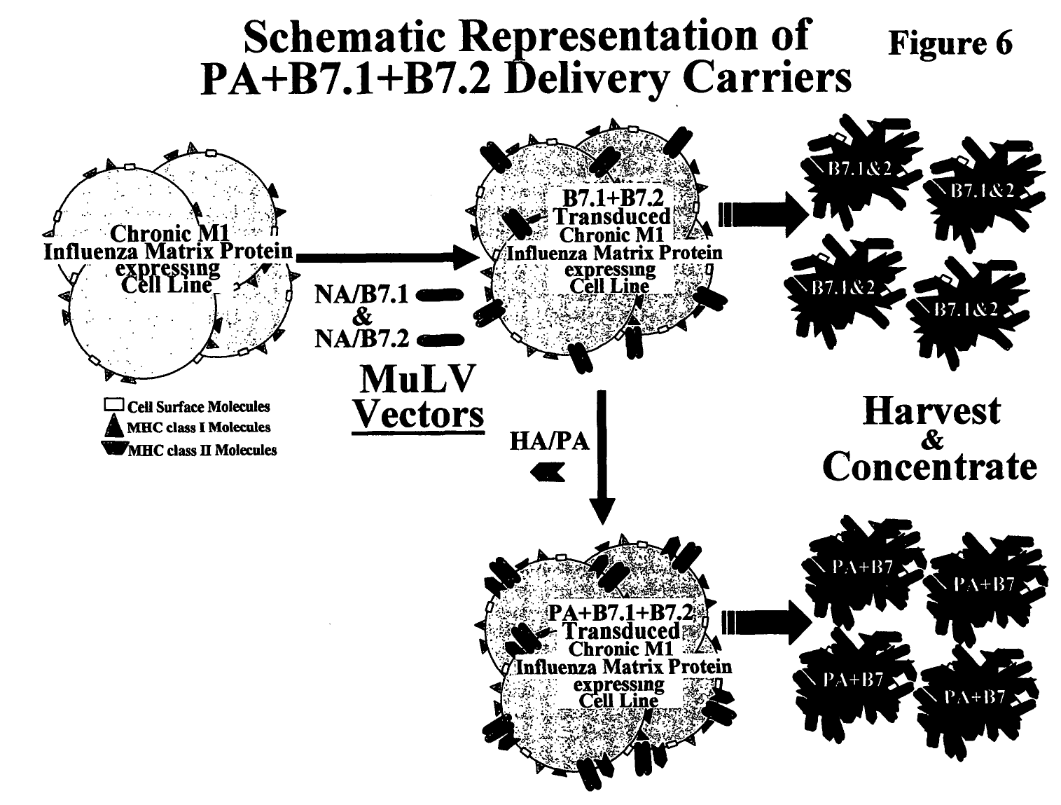

FIG. 7 is a schematic representation for using virus-like-particle release to incorporate the Protective Antigen (PA) protein from anthrax into a particle structure for diagnostic purposes. The PA containing particles could be used as an anthrax specific antigen for the purification of this specific viral antigen and/or nucleic acids, in addition to an antigen preparation for detection of immune responses against anthrax exposure in humans by the analysis for the presence of antibodies. Detection could be by ELISA, Western Blot and/or immune fluorescence, but are not limited to these systems. The major advantage of incorporating a protein of interest (here the PA protein as an example) into a particle is for the purification of the antigen using standard generic techniques for isolating particles (viral or non-viral) that could be preformed by one skilled in the art for an antigen in the proper orientation, conformation, configuration, multimerization, and post-translational modifications that include but not limited to glycosylation, polyADPribosylation, and myristylation to improve the functional parameters associated with the said molecule(s).

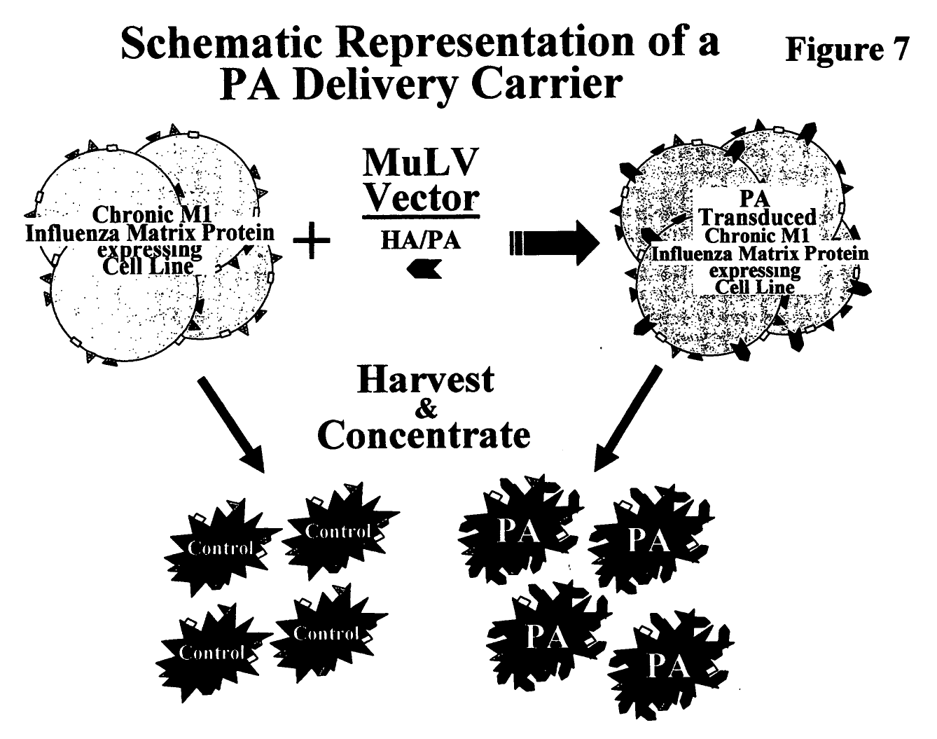

### DETAILED DESCRIPTION OF THE PREFERRED EMBODIMENTS

The present invention relates to the use of particles to capture and incorporate recombinant molecules, for the in vitro use of these particles, and for the in vivo delivery of these particles within a mammalian host as a universal protein delivery system. The invention describes a process and the utility of that process to develop therapeutic entities and diagnostic reagents that can protect, enhance, suppress, alleviate, repair, differentiation, detect, and modulate cellular processes by the delivery of recombinant molecules to influence these cellular processes in vivo and in vitro.

In the preferred embodiment, the particles used in the invention are produced in vitro from cells genetically engineered to express recombinant molecules onto their cells' surface and genetically engineered to produce budding particles that capture and incorporate these expressed recombinant molecules such that the particles when harvested contain the recombinant molecules. In accordance with the invention, the particles are virus-like-particles and as such are not infectious, but rather serve as biological carriers of expressed recombinant molecules that are removed from the cells' surface as the particle is released from the cell. Such particles could be harvested and then used as recombinant molecules in vitro and in vivo in accordance with the invention.

The invention provides for the use of the recombinant molecule(s) containing particles to present the relevant molecule(s) to various biological processes, for example, as an immunoprophylactic or immunotherapy to treat cancer, exposure to toxins, infectious diseases and as an alternative to conventional drug and antibiotic therapies, especially in cases where resistance has developed. Pursuant to the present invention, molecules have been expressed and/or induced on the surface of the continuously expressing particle-producing host cell line, and the released particles are harvested. The recovered particles present transduced or endogenously expressed antigen(s) together with co-stimulatory molecule(s) directly to the immune system, or are picked up by “professional” antigen presenting cells (APCs), such as dendritic cells and macrophages, for presentation to lymphoid cells. The minimum requirement of an APC for activation of T-lymphocytes are to degrade complex protein antigens into antigen fragments, to present these antigen fragments that were bound to MHC molecules present on the particles by virtue of their presence on the host cell surface and subsequently captured and incorporated into particles along with the recombinant expressed co-stimulatory molecules, like B7.1 and B7.2.

Other examples of how the invention provides for the use of the recombinant molecule(s) containing particles to present the relevant molecule(s) to various biological processes can be shown as the use of the invention as a bio-scavenging enzyme or receptor, as a cytokine, growth factor, chemo-attractant, and/or a generalized protein, peptide, antibody delivery system in vitro or in vivo to influence various biological processes. Pursuant to this embodiment of the invention, molecules have been expressed and/or induced on the surface of the continuously expressing particle-producing host cell line, and the released particles are harvested. The recovered particles contain the transduced or endogenously expressed recombinant molecules that were expressed on the surface of the continuously expressing particle-producing host cell line, and the released particles are harvested. The recovered particles contain transduced or endogenously expressed recombinant molecules and these particles could be used directly. In vitro, the particles could be used to stimulate biological processes such as cell growth, expansion, and differentiation—for example by containing a factor that maintains growth and expansion of a particular cell type or line without differentiation, or inducing differentiation at the expense of growth. This could be the case for embryonic and progenitor stem cells. In addition, the particles could be used in vitro to purify specific molecules in active and native forms or as a reagent for the detection and analysis for the presence of antibodies or as a control or standard for assays involving the captured and incorporated recombinant molecule. In vivo, recombinant made particles could be used as a generalized protein delivery system capable of delivering proteins, peptides, and antibodies to scavenge toxic molecules endogenous to human systems or those exogenously introduce—for example derived from bio-terrorism actions. Also attracting cells to the region that would assist in the repair process could use in vivo use of the invention to deliver necessary factors to influence the differentiation and repair of cellular processes involved in cellular and/or tissue regeneration by interacting directly with the repair process or by influencing the repair process. In vivo, the particles could be used to deliver recombinant molecules that influence cellular responses—for example suppress immune responses during bone marrow transplantation, influence T-lymphocyte population expansion that may result in a favorable response in certain disease conditions and to delivery recombinant molecules to relieve disease symptoms.

Techniques and terms for transduction, sequence isolation, in-frame fusions or ligation, gene, recombinant, coding of sequences, intracellular-transmembrane domains, ecto- or extracellular portions of proteins, genetically-modified, virus-like-particles, virus infection, inactivation of virus particle preparations, viral budding, digestion or restriction enzyme analyses, in addition to other molecular biologic or molecular virology techniques and terms that are established and used in the art are described in standard laboratory manuals and references, such as, for instance, Sambrook et al., MOLECULAR CLONING, A LABORATORY MANUAL, 4^{nd} Ed.; Cold Spring Harbor Laboratory Press, Cold Spring Harbor, N.Y.

Pathogens against which the present invention may be applicable in the formation of biological particles containing pathogen antigen(s) include, but are limited to bacteria, parasites, protozoa, fungi, prion, and viruses. Viruses are infectious agents (pathogens) including hepatitis A, hepatitis B, hepatitis C, herpes simplex viruses, varicella zoster, Epstein-Barr virus, cytomegalovirus, human herpesvirus-6, -7, -8, HIV-1, HIV-2, HTLV-1, HTLV-2, Rubella, Rubeola, Influenza, Rotavirus, West Nile, Dengue and other emerging flaviviruses. Due to bio-terrorism, Category-A biological diseases as define by the CDC, which include: Bacillus anthracis (anthrax), Clostridium botulinum toxin (botulism), Yersinia pestis (plague), Variola mayor (smallpox), Francisella tularensis (tularemia) and pathogens responsible for viral hemorrhagic fevers are included. Prions are the transmissible pathogenic agents responsible for diseases such as scrapie, bovine spongiform encephalopathy, and associated human diseases. Fungi, protozoa and parasites include Toxoplasma, trypanosomes, babesia, rickettsia, malaria, and enteric pathogens. Bacteria include species of Chlamydia, Helicobacter, Neisseria, Mycobacteria, (especially M. tuberculosi). The scientific literature identifies 1,415 species of infectious organism known to be pathogenic to humans, including 217 viruses and prions, 538 bacteria and rickettsia, 307 fungi, 66 protozoa and 287 helminthes. Out of these, 868 (61%) are zoonotic, that is, they can be transmitted between humans and animals, and 175 pathogenic species are associated with diseases considered to be “emerging” are included. Over 100 viruses have been associated with acute central nervous system infections, causing among other diseases encephalitis and meningitis; Nipah virus in Malaysia and neurovirulent enterovirus (70 strains) that cause severe neurological disease; vector borne disease agents include Japanese encephalitis, Barmah Forest, Ross River, and Chikungunya viruses; hendra virus, formerly called equine morbillivirus a rabies-related virus, Australian bat lyssavirus, and a virus associated with porcine stillbirths and malformations, Menangle virus. Most emerging viruses are zoonotic and because of the large number of present and emerging pathogens that infect human are zoonotic, veterinary viral-delivered vaccinology strategies are also encompassed within the scope of the invention.

Antigens against which the present invention may be applicable in the formation of particles containing recombinant forms include polypeptides encoded by the pathogen listed above. The multitudes of antigens encoded by these agents that may be expressed include, but are not limited to external surface proteins and structure proteins including enzymes, transcription factors, and other cell regulatory proteins. For example, antigens encoded by any genes of the HIV-1 genome including gag, pol, vif vpr, vpu, tat, rev, env, and nef may be all present as either intact antigens or immune dominate peptides. Another example is the pathogenic prion protein (PrPSc) template and endogenous cellular prion protein (PrPC). Proteins include all known and to be discovered gene or nucleic acid containing encoded proteins, cytokines and related molecules such as interleukins, growth factors, chemokines, adhesion molecules, neurotrophic factors, MMPs/TIMPs, receptors, and developmental proteins. Peptides include any amino acid sequence that could be made and/or found in nature; expressed as monomers or as oligomeric versions, including immune-dominant epitopes. Antibodies include polyclonal and monoclonal derived against any human, mammalian, bacterium, parasite, protozoa, fungi, prion, and/or virus antigen. In addition, tumor antigens are included in the scope of this invention. Two types of antigens have been identified on tumor cells: Tumor-specific transplantation antigens (TSTAs) that are unique to cancer cells, and tumor-associated transplantation antigens (TATAs) that are found on both cancer and normal cells. Thus, tumor antigens consist of TSTAs, TATAs, and oncogene proteins. Tumor-specific antigens have been identified on tumors induced by chemical and physical carcinogens and some virally induced tumors. The antigen(s) can be present within the chronic expressing pathogen containing cell line that is used as the particle-producing host or as part of an infectious process, naturally native to the cell, transduced or transfected by biological (viral vectors), chemical (liposomes), or mechanical (electroporation) methods. The pathogen antigen could be expressed and assembled into the pathogen itself, or associated with a different pathogen particle.

The following examples further illustrate experiments that have demonstrated reduction to practice and utility of selected preferred embodiments of the present invention, although they are in no way a limitation of the teachings or disclosure of the present invention as set forth herein.

### EXAMPLE 1

### As A Bio-Scavenging Enzyme

Production and formation of hepatic cell line expressing mammalian butyrlcholinesterase and the incorporation of said molecule into virus-like-particles and the utility of these particles to prevent or diminish toxicity in vivo demonstrates the principle of this invention.

Butyrlcholinesterase hydrolyze organophosphates, a major component in nerve gas and has been shown to protect cells in vivo from bio-terrorism and drug-induced toxicity. A hepatoma cell line was used to stably express on its cell surface, using murine leukemia virus vectors, the butyrlcholinesterase gene. The heptoma cell line was either subsequently virally infected or contain one or more components (viral, non-viral, or innate) that resulted in the continuous budding of particles from the said cell line. The harvesting of the particles can be done by ultracentrifugation or preferably by selective dehydration and precipitation (use of polyethylene glycol or similar agents), affinity and/or size dependent chromatography. The particles would be tested for butyrlcholinesterase biological activity in vitro. Optimal detoxification activity by butyrlcholinesterase requires the tetrameric form of the enzyme that is enhanced by the co-expression of a proline-rich attachment domain as a peptide coding the proline-rich attachment domain (PRAD) within the said particle-producing cell. Retention times of recombinant appropriate post-translational modified butyrlcholinesterase could be compared to observed retention times of purified butyrlcholinesterase from native serum sources. Various routes of delivery will be explored including intravenous, intraperitoneal, intramuscular via autoinjectors, and pulmonary/intranasal via “puffer” devices at different doses to establish optimal bioavailability and retention times. In addition to biowarfare, the recombinant particles may be used to alleviate succinylcholine-induced apnea and to treat cocaine or other drug overdosed individuals.

### EXAMPLE 2

### As A Bio-Scavenging Receptor

Production and formation of cell lines expressing mammalian Tumor Necrosis Factor Receptor II and the incorporation of said molecule into a virus-like-particle and the utility of these particles in binding, sequestering and elimination from a biological system demonstrates the principle of this invention.

This example relates to bio-scavenging ability of the invention to scavenge the cytokine TNF-alpha using particles that incorporated the TNF receptor II molecule. Cell lines would be established expressing the said molecule onto their cell surface. The cell line could be either subsequently virally infected or contain one or more components (viral, non-viral, or innate) that resulted in the continuous budding of particles from the said cell line. The harvesting of the particles can be done by ultracentrifugation or preferably by selective dehydration and precipitation (use of polyethylene glycol or similar agents), affinity and/or size dependent chromatography. The particles would be tested for their ability to bind and sequester TNF-alpha biological activity in vitro, followed by in vivo testing using an arthritic-induced animal model (BalbC SCIDs). Successful demonstration in animal models could lead to clinical trials in humans.

### EXAMPLE 3

### As A Cytokine and/or Growth Factor Involved in Stimulating Biological Processes and/or Cellular/Tissue Repair and/or Attracting Specific Cell Types to Enhance the Repair Process In Vivo

Production and formation of cell lines expressing mammalian cytokines and/or growth factors either known or yet to be discovered and the incorporation of said molecule(s) into virus particles by acute infection of cells harboring said molecules or virus-like-particles, and the utility of these particles to influence cellular and/or biological properties, including but not limited to receptor binding demonstrates the principle of this invention.

This example relates to the incorporation and use as a delivery system in vivo of molecules required or capable of influencing and/or enhancing cellular and tissue regeneration. Cell lines are established expressing the genetically modified molecule(s) to be expressed on the said cells' surface using techniques established within the art and the incorporation or association of these molecules with viral or non-viral, infectious or non-infectious particles. These particles could be delivered to a human or mammalian host by oral, suppository, intravenous, intra-muscular, inter-cranial, inter-peritoneal, or directly into organs, capillaries, ducts, or lymphoid system either alone or associated with biological or non-biological materials or devices. An inter-respiratory device, cutaneous and topical applications, aerosols, creams, puffers, or on surfaces could be envisioned for particle delivery systemic or local. Surfaces include, but not limited to, synthetic, non-synthetic, biological, or nonbiological matrixes including autologous, allogeneic, and xenogeneic extracellular matrix materials. Dependent on the biological molecule delivered, the delivery route could be tested for efficacy in animal models followed by clinical trials in human.

### EXAMPLE 4

### As A Cytokine and/or Growth Factor Involved in Stimulating Biological Processes and/or Cellular/Tissue Repair and/or Attracting Specific Cell Types to Enhance the Repair Process In Vitro

Production and formation of cell lines expressing mammalian cytokines and/or growth factors either known or yet to be discovered and the incorporation of said molecule(s) into non-viral, virus or virus-like-particles and the utility of these particles to influence cellular and/or biological properties in culture in vitro, including but not limited to receptor binding demonstrates the principle of this invention.

This example relates to the incorporation and use as a delivery system in vitro of molecules required or capable of influencing and/or enhancing cellular processes including cellular differentiation and/or human and mammalian oocyte activation and the influence towards blastocyst formation, embryonic stem cell establishment, and/or the self-renewal and/or differentiation of that stem cell line into specific lineages. Cell lines are established expressing the genetically modified molecule(s) to be expressed on the said cells' surface using techniques established within the art and the incorporation or association of these molecules with viral or non-viral, infectious or non-infectious particles. These particles could be introduced to the culture fluids of cells to influence cellular processes.

### EXAMPLE 5

### As A Protein, Peptide, and/or Antibody in vivo Delivery System Involved in Influencing Biological Processes

Production and formation of cell lines expressing mammalian proteins, peptides, and/or antibodies either known or yet to be discovered and the incorporation of said molecule(s) into non-virus, virus or virus-like-particles and the utility of these particles to influence biological properties, including but not limited to therapeutic host delivery demonstrates the principle of this invention.

This example relates to the incorporation and use as an in vivo delivery system, molecules required or capable of influencing biological properties. Properties could include the incorporation of amino acid sequences for proteins and/or antibodies, or of peptides as monomers or multimers to influence biological properties by inhibiting or stimulating specific biological events, including but not limited to apoptotic and anti-apoptotic factors. Cell lines are established expressing the genetically modified molecule(s) to be expressed on the said cells' surface using techniques established within the art and the incorporation or association of these molecules with viral or non-viral, infectious or non-infectious particles. These particles could be delivered to a human or mammalian host by oral, suppository, intravenous, intramuscular, inter-cranial, inter-peritoneal, or directly into organs, capillaries, ducts, or lymphoid system either alone or associated with biological or non-biological materials or devices. An inter-respiratory device, cutaneous and topical applications, aerosols, creams, puffers, or on surfaces could be envisioned for particle delivery systemic or local. Surfaces include, but not limited to, synthetic, non-synthetic, biological, or non-biological matrixes including autologous, allogeneic, and xenogeneic extracellular matrix materials. Dependent on the biological molecule delivered, the delivery route could be tested for efficacy in animal models followed by clinical trials in human.

### EXAMPLE 6

### Establishing a Universal Particle Expressing Cell Line for Delivery of any Recombinant Protein, Peptide, and/or Antibody for Therapeutic and/or Diagnostic Purposes

The principle of this invention could be further demonstrated by in vivo experiments in mice, non-human primates, and ultimately clinical trials/treatments in humans.

In the present invention, a virus-based protein delivery system can be engineered to serve as a potential vaccine candidate against infectious diseases and cancer. We believe that this system can serve as a general delivery system in vivo for any recombinant protein, peptide, and/or antibody based therapeutic, opening up the potential to any and all disease conditions. To insure safety we have be inactivating and thereby destroying infectivity of infectious virus particles. As a further precaution, we are using virus-like-particles that are innately devoid of the ability to infect cells. The present example uses influenza virus matrix protein as our core protein to induce a budding process to produce virus-like-particles, but other single virus components or combination of components could be envisioned.

The Influenza matrix protein similar to the matrix proteins of retroviruses, vesicular stomatitis virus, and human parainfluenza virus type 1 has intrinsic budding activity, and when expressed alone, will bud particles into the culture supernatant. We expect these budding particles to “capture” cell surface expressed recombinant proteins. Although the interactions between the internal viral component and the cytoplasmic tail of external viral proteins are not an absolute requirement for particle formation, chimeric constructions of hemagglutinin—HA and neuramimidase—NA intracellular-transmembrane domains fused in-frame with heterologous extracellular portions of proteins could further enhance incorporation of recombinant molecules within the released particles. Since the FDA already approves live attenuated influenza virus vaccines for human administration, using the matrix proteins from influenza virus as our universal particle producing cell line should offer a safe and efficacious method to deliver recombinant proteins, peptides, and/or antibodies-based therapeutic to humans.

To establish a M1 Influenza-A matrix protein expressing cell line, RNA would be isolated from Influenza-A infected MDCK cells, reversed transcribed, PCR amplified, and the full length M1 matrix protein cloned into a PCR cloning vector. Oligonucleotides will be synthesized encompassing the 5′ and 3′ ends of the 759 nucleotide M1 matrix protein (GenBank Accession AF222823) and the cloned fragment will be synthesized to confirm the accuracy of the amplified fragment. The M1 gene will be transferred into a truncated HIV-LTR clone, pJM167, containing a minimal tat-inducible promoter. Past experience has demonstrated the tat-inducible promoter, in the presence of tat (pJM310) to result in high protein expression driven by the continuous transcriptional activation by tat binding to the TAR element within the HIV-LTR. Once the M1 gene fragment is cloned behind the truncated LTR, this plasmid will be co-transfected with pJM310 into a series of T- and B-lymphocytic cell lines (Suptl, Hut78, Raji, Molt-3) by clectroporation (to gain the highest number of integrated gene copies) of the cells in the presence of media containing the plasmids. The cell lines will be initially screened by RT-PCR for M1 matrix protein expression and the line with the highest signal will be single cell cloned. Evidence of vesicular particles in clarified concentrated supernatants of cultured cells will be confirmed by negative staining electron microscopy particle counts.

### EXAMPLE 7

### As An Immune Regulatory

Production and formation of cell lines expressing mammalian proteins, peptides, and/or antibodies either known or yet to be discovered and the incorporation of said molecule(s) into non-virus, virus or virus-like-particles and the utility of these particles to influence immune responses, including but not limited to therapeutic host delivery demonstrates the principle of this invention.

This example relates to the incorporation of immune modulator molecules into cell lines that express particles that capture and incorporates said molecules. These immune molecules could include one or more of the following proteins, but are not limited to these molecules—B7.1, B7.2, CTLA-4, OXA40, 4-IBB, CD27—that are involved in the activation or suppression of immune responses. In addition to these molecules in some situation, specific antigens to infectious disease agents, cancer, and/or autoimmune diseases would be included into the release particles by their inclusion on to the host cells' surface. In addition to the incorporation of immune and antigen molecules that were exogenously expressed on the cells' surface by standard molecular biological techniques, native cellular expressed molecules are expected to be co-incorporated into the released particles. These molecules would include processed peptides from the exogenously expressed antigens within the groove of MHC class I and class II, plus CD1 molecules. The processed peptides and glycolipids associated with MHC and CD1 molecules, respectively, would stimulate immune responses by binding to the CD3 molecule and the T-cell receptors of appropriate cells, while the immune modulator molecules will interact through there respective ligands or receptors. Although the mechanism of these approaches might be induction or repression of immune responses through the humoral and cell-mediated arms of the immune system, other mechanisms may be implored to affect immune modulation that may involve but not limited to the expression of cellular factors that influence immune responses. One example is the expression of a CD8+ cell factor that can inhibit HIV-1 expression in some HIV-infected individuals.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth.

1. A process for production and formation of a generalized in vivo delivery system that incorporates cell surface expressed recombinant amino acid containing sequences and innate host cell surface cellular molecules within a biologically formed particle, comprising of viral components that bud from host cells that are genetically modified to express cell surface recombinant molecules.

2. The process of claim 1 wherein said generalized in vivo delivery system deliver amino acid containing sequences that are protein(s), peptide(s), and/or antibodies.

3. The process of claim 2 wherein the said proteins, peptides, and/or antibodies are expressed on the surface of host cells that produced the budding particles of claim 1.

4. A method for establishing host cells of claim 1 comprising of viral components that result in budding particles.

5. The process of genetically modifying said host cells of claim 4 such that the budding particles contain the said proteins, peptides and/or antibodies to be delivered in claim 2.

6. The process of claim 5 wherein the proteins, peptides, and/or antibodies are passively incorporated into the budding particles by their proximity within the membrane of host cells.

7. The process of claim 5 wherein the proteins, peptides, and/or antibodies are passively incorporated into the budding particles by inclusion of transmembrane sequences onto the coding sequence of said proteins, peptides, and/or antibodies resulting in membrane expression on host cells.

8. A method by which the viral components of claim 4 are viral matrix proteins.

9. A method of claim 8 where the virus is influenza.

10. The process of claim 7 wherein the proteins, peptides, and/or antibodies are actively incorporation into budding particles by matching viral matrix proteins to the intracellular domains of viral envelope proteins.

11. The process of claim 1 wherein the budding particles are non-infectious, concentrated, and formulated as a drug for administration to a mammalian recipient.

12. A process of claim 11 wherein treating a mammalian recipient to provide in said recipient a therapeutic response, comprising administering to a mammalian recipient non-infectious budding particles in an amount effective to be therapeutic.

13. A process of claim 12 where the non-infectious particles were either previously infectious (containing viral genome) and inactivated or produced from virus-like-particles (missing one or more viral genome components required for infectious virus formation).

14. A process of claim 12 where the therapeutic response can induce signal transduction pathways by cell surface receptor engagement; induce or inhibit cellular differentiation; stimulate or suppress immune responses; attract or repel cells; prevent host cell and organ toxicity.

15. A process of claim 14 where the therapeutic response is a vaccine or a prophylactic.

16. A process of claim 12 where the mammalian recipient is a human.

17. A process of claim 12 where the mammalian recipient is a non-human primate, canine, feline, or other non-human mammalian species.

18. A process of claim 1 wherein said host cells are obtained from a cancer recipient.

19. A process of claim 1 wherein said host cells are obtained from a transplant recipient.

20. A process of claim 1 wherein said host cells are an established cell line.

21. A process of claim 1 wherein said host cells are MHC matched.

22. A process for treating a transplant recipient to reduce in said recipient an immune response to an alloantigen, comprising treating the transplant organ with a therapeutically effective amount of the budding particles of claim 11, wherein either the budding particles are derived from host cells obtained from the donor and/or the budding particles are derived from host cells containing cell surface expressed amino acid containing sequences that code for molecules that can reduce immune response against the alloantigen.

23. A process for treating a mammalian subject for promoting connective tissue growth, resulting in tissue engineering comprising treating a recipient in need of connective tissue growth by administering a therapeutically effective amount of the budding particles of claim 11, wherein the budding particles are derived from host cells containing incorporated amino acid sequences that code for molecules that can either promote healing or attract endogenous cells that can promote healing.

24. A process for treating a mammalian subject for prevention of toxicity of biological or chemical agents; comprising treating a recipient exposed or in possibility of exposure by administering a therapeutically effective amount of the budding particles of claim 11, wherein the budding particles are derived from host cells containing incorporated amino acid sequences that code for molecules that can act as a bio-scavenger to either remove or inactivate the toxic substance.

25. A process for treating a mammalian subject for therapeutic purposes by administering budding particles of claim 11 derived from host cells that display therapeutic moieties as an amino acid sequence where the particles behaved as a drug delivery vehicle.

26. A process of claim 1 where amino acid containing diagnostic molecules are expressed on the surface of host cells that produce particles; these particles can be used as diagnostic reagents within diagnostic test kits.
